(19) United States
(12) Patent Application Publication
(10) Pub. No.: US 2004/0167449 A1
(43) Pub. Date: Aug. 26, 2004

(54) Appliance for lordosis adjustment for treating discomfort in, or originating in, the cervical spine region

(76) Inventors: Dan S. Heffez, Evanston, IL (US); Gene P. Bernardoni, Chicago, IL (US)

Correspondence Address: SCHIFF HARDIN, LLP, PATENT DEPARTMENT, 6600 SEARS TOWER, CHICAGO, IL, 60606-6473, US

(21) Appl. No.: 10/375,739

(22) Filed: Feb. 26, 2003

(51) Int. Cl. A61F 5/00

(52) U.S. Cl. 602/19

(57) ABSTRACT

A body-worn appliance for lordosis adjustment of a patient to alleviate pain or discomfort in or originating in the cervical spine region of the patient has an anterior portion and a posterior portion connected together by adjustable straps. The anterior portion has a rigid frame with a central opening, and the posterior portion has a relatively short height so that, when worn by a patient, the appliance has minimal bracing or supporting effect. One or more pads is/are releasably attachable to the posterior portion to set a thickness of the combination of the posterior portion and the pad or pads. Setting of this thickness is undertaken to effect a desired lordosis adjustment of the patient when the appliance is worn by the patient, so as to produce a neutral cervical spine orientation, thereby alleviating discomfort in or originating in the cervical spinal region without the necessity of wearing a neck brace or support.

### BACKGROUND OF THE INVENTION

1. Field of the Invention

The present invention is directed to an appliance, of the type to be worn by a person, for effecting lordosis adjustment in order to treat discomfort suffered by the person IN or originating in the cervical region.

2. Description of the Prior Art

Various types of body-worn braces or support are known for providing mechanical assistance to aid weak or ineffectual muscles or the treatment of the various types of back disorders and discomfort. Examples of such known orthotic braces are described in U.S. Pat. Nos. 5,911,697; 5,718,670; 5,690,609; 5,547,462; 5,433,697; 5,295,947; 5,259,831; 4,285,336 and 2,813,526.

These devices, and others of their type, have in common a design and structure that are specifically adapted to provide some type of mechanical support, primarily in the lumbar region, which would normally be provided by a healthy muscular or skeletal system, but which in certain occasions, due to disease or injury, is lacking or in need of augmentation. In general, these types of known devices can be considered as “stiffening” the lumbar region for the purpose of achieving this result.

Certain of these devices also are specifically designed to constrict or confine movement of the lumbar region for cases where such movement induces pain or discomfort, or aggravates the particular back disorder being treated.

In a healthy person with good posture, the spinal column exhibits a curvature toward the abdomen (anterior curvature) as well as a curvature in the upper back region and into the neck region. The normal concave curvature of the lumbar spine and cervical spine column is referred to as lordosis. Because of the interconnectedness of the vertebrae forming the spinal column, it is known that abnormal curvature of the spinal column in one region can produce abnormal curvature in another region.

The known braces or supports of the type described above, by stiffening or confining the lumbar region, may have an incidental effect on lumbar lordosis, but they are not designed to intentionally adjust or redirect lumbar lordosis as their primary function. Moreover, conventional braces and supports of the above type are designed for treating particular disorders of the lumbar region and are not designed to intentionally effect or alter other regions of the spinal column, such as the cervical spine region.

Many supports and braces are also known for providing similar mechanical stiffening and confinement in the cervical spine region. The conventional approach for patients suffering from neck pain or other neck disorders has been to prescribe the use of such neck or cervical spine braces, which interact with the patient directly in the cervical spine region.

### SUMMARY OF THE INVENTION

It is an object of the present invention to provide an appliance to be worn by a patient suffering from pain or discomfort in, or originating in, the cervical spine region for alleviating the pain or discomfort without the necessity of using a conventional neck/cervical spine brace.

The above object is achieved in accordance with the principles of the present invention in an appliance adapted to be worn by such a patient in the lumbo-sacro region which has a design and structure specifically for effecting lordosis adjustment in the lumbo-sacro region in a manner which has the consequence of also causing lordosis adjustment in the cervical spine region, thereby alleviating the cervical spine discomfort or pain experienced by the patient.

The present invention is based on the recognition that by making a lordosis adjustment in the lumbo-sacro region, by the application of force vectors to the back and abdomen at specified locations, in order to replicate as closely as possible normal, healthy lordosis, this will necessarily result in the spinal column in the cervical spine region also exhibiting a lordosis restored to that of a healthy person with good posture, or at least will significantly improve lordosis in the cervical spine region. The appliance adapted to be worn by the patient in the lumbo-sacro region has a structure and design for producing the aforementioned force vectors at specified locations in order to restore healthy lordosis for the purpose of alleviating discomfort in the cervical spine region. The necessity of wearing a discomforting and a cumbersome neck or cervical spine brace is therefore avoided. Moreover, because the appliance for lordosis adjustment is not intended to be a brace or support of the conventional type described initially, its structure and bulk are relatively minimal and lightweight, and therefore it can be worn in a relatively unobtrusive manner under clothing.

The appliance includes an anterior portion with a large central opening therein and a surrounding frame that is adapted to be worn approximately over the abdomen. This anterior portion of the appliance is connected by straps to a posterior portion which is designed to be relatively short in vertical height to cover only approximately the L2 through L4 vertebrae (the peak lordotic curve occurs at the L3 vertebra). The short vertical height of the posterior portion of the appliance is possible because it is not designed or intended to perform a bracing function.

The aforementioned openness of the anterior portion is important for achieving the intended lordosis adjustment. If the anterior portion of the appliance were solid, i.e., without a central opening, the abdominal resistance would be too large and the force vectors produced by the posterior portion of the appliance would have little or no effect.

The degree of lordosis adjustment is created by one or more pads that are removably attached to and carried by the posterior portion of the appliance. It would be possible for the posterior portion of the appliance to be customized to have a specified curvature adapted to a particular patient to, at least to a certain extent, produce the desired amount of lordosis adjustment for that patient. In order to make the appliance more universally applicable for a large number of patients, however, a standardized posterior portion, or at least only a few standardized posterior portions with respectively different curvatures, is/are employed, and the amount of lordosis adjustment is achieved by adding pads to this standardized posterior portion, as needed. The more pads that are added, the greater the amount of lordosis adjustment that can be achieved. The pads can be attachable one on top of the other by a hook-and-loop system, or by any other appropriate system allowing temporary attachment. In general, the posterior portion itself will provide only a slight lordosis adjustment, and it is the use of one or more pads which is intended to produce the prescribed amount of lordosis adjustment for alleviating the particular discomfort in the cervical spine region of a given patient.

The anterior portion of the appliance, by virtue of its shape and extent and its open center, is intended to apply force vectors at two basic impact regions. A lower point of impact is approximately at the symphisis pubis, and the upper point of impact is in the lower costal margin, i.e., approximately at the lower edge of the rib cage. The force vectors applied in the lower costal margin are preferably applied at two impact points which are spaced from each other, so that a total of three impact points, approximately located at respective vertices of a triangle, are produced by the anterior portion of the appliance.

As noted above, a benefit of the appliance is that it does not necessarily need to be customized to the anatomy of the person by whom it will be worn, since the desired lordosis adjustment is achieved by the appropriate selection and use of one or more pads attached to the posterior portion of the appliance. Nevertheless, the appliance can be manufactured in several basic sizes, such as a size adapted to be worn by a child and a size adapted to be worn by an adult. Intermediate sizes also could be available, if needed.

### DESCRIPTION OF THE DRAWINGS

FIG. 1 is a perspective view of a first embodiment of an appliance for lordosis adjustment in accordance with the invention, showing one additional pad for attachment to the posterior portion.

FIG. 2 illustrates a pad for use in a second embodiment of an appliance for lordosis adjustment in accordance with the invention.

FIG. 3 schematically illustrates normal spinal column lordosis which represents a target to be achieved by the inventive appliance to relieve neck discomfort.

FIG. 4 is a sectional view illustrating the forces generated when the inventive appliance for lordosis adjustment is worn by a patient as shown in FIG. 3.

FIG. 5 illustrates the lordosis adjustment achieved by a seated person wearing an appliance in accordance with the invention.

FIG. 6 illustrates spinal column lordosis (or absence thereof) as would occur in the seated person not wearing the inventive appliance.

FIG. 7 is a schematic axial section through the lumbar region of a person wearing an appliance in accordance with the invention.

### DESCRIPTION OF THE PREFERRED EMBODIMENTS

The lumbo-sacro support in accordance with the invention is specifically designed to maintain or accentuate the lumbar lordosis in order to promote a neutral orientation of the head or neck on the upper body, for the purpose of alleviating neck pain or discomfort, or in general pain or discomfort in, or originating in the cervical spine region without the necessity of wearing a neck brace or support. The promotion of the proper posture of the head or neck on the upper body is achieved by appropriate lordosis adjustment in the lumbar region through the action of the standing and righting reflexes which normally maintain proper erect body posture. The importance of such a neutral neck or cervical spine position, as opposed to a position of flexion or extension, follows from the relationship between the nerve roots and the spinal cord to the bony spinal canal and neural foramina. The anteroposterior dimension of the spinal canal and neural foramina changes with the orientation of the neck. Mild spinal flexion enlarges the anteroposterior diameter, while neck extension narrows the anteroposterior diameter. In cases of narrowing of the spinal canal (stenosis) such changes in the diameter of the spinal canal can determine whether the spinal cord or the nerve roots are compressed, irritated or traumatized, as well as determining the extent of such compression, or irritation or trauma. Maintaining a neutral cervical spine posture can minimize spinal cord or nerve root irritation, and in some cases eliminate it completely. Poor lumbar and thoracic posture necessitates compensatory cervical alignment in order to allow the person experiencing the poor posture to be able to look straight ahead.

Improper cervical lordosis accentuates cervical extension, and as a result narrows the spinal canal and neural foramina. By encouraging and ensuring proper lumbar lordosis in the sitting or standing position, the maintenance of a neutral neck orientation is promoted and therefore is useful in the treatment of conditions that result in or from spinal canal narrowing. Similarly, treatment or manipulation of the lumbar spine designed to maintain proper lumbar lordosis is expected to promote neutral cervical alignment and orientation, and can be beneficial to the management of conditions that result in or from spinal canal narrowing.

Examples of conditions which can be effectively treated or managed by the use of the inventive appliance to optimize cervical posture are as follows:

Congenital cervical spinal stenosis which does not warrant surgical decompression.

Cervical spinal stenosis secondary to the protrusion of bone parts, bulging or herniated intervertabral disc, thickened ligamentum flavum or ossified posterior longitudinal ligament which does not warrant surgical decompression.

Following cervical spine surgery, when maintaining proper posture is important for the prevention of secondary spinal deformity or intervertebral disc degeneration.

Arthritides which damage the joints and supportive structures of the spine and that might contribute to poor cervical posture.

Cervical myelopathy due to cervical spinal cord compression when the spinal cord compression is of clinical significance, primarily when the neck is positioned in extension, and when neither the severity of myelopathy or spinal cord decompression warrants surgical intervention.

Cases of fibromyalgia syndrome, when these are associated with findings of myelopathy and mild spinal stenosis.

Cases of the Chiari I malformation in which neck extension would provoke an accentuation of compression of the brain stem and spinal cord secondary to increased herniation of the cerebellar tonsils through the foramen magnum at the base of the skull.

Cervical radiculopathy or nerve root irritation due to stenosis of a neural foramen if the stenosis is of clinical significance, primarily when the patient's neck is in extension and when surgical decompression is not required.

Examples of symptoms which may arise from the above-mentioned anatomical or pathological conditions, and therefore may be amenable to a treatment strategy involving optimizing cervical spine posture using the inventive appliance, as are follows:

Headache, especially occipital, vertex and retroocular headaches—cervicogenic headaches.

Pain including neck, interscapular, subscapular, midline spinal, lumbar, arm or leg pain.

Certain facial pains including pains in the distribution of temperomandibular joint, when believed to be of cervicogenic origin.

Dizziness or vertigo of spinal origin.

Limb or facial numbness or tingling.

Impaired balance when standing or walking.

Disturbance of bowel motility including diarrhea and constipation and irritable bowel syndrome when diagnosed as being of neurogenic, spinal origin.

Urinary frequency, urgency or incontinence when diagnosed to be of neurogenic cervical spinal origin.

Physical muscular fatigue when diagnosed as arising due to cervical spinal cord compression or irritation; such fatigue is frequently described by patients with fibromyalgia, chronic fatigue syndrome and cervical myelopathy.

Otological symptoms such as tinnnitus, hyperacusis, or pressure in the ears, diagnosed to be of cervicogenic origin.

Autonomic disturbances such tachyarrythmias, hypertension, hypotension, impaired circulation in the hands and feet, Postural Orthostatic Tachycardia Syndrome and Neurally Mediated Hypotension, if diagnosed to be due to a cervical neurogenic mechanism.

Blurred or double vision diagnosed to be of a cervical neurogenic origin.

Limb weakness diagnosed to be of a neurogenic, cervical spinal origin.

In general, symptoms as exemplified above can be characterized as symptoms arising from spinal cord and/or nerve root compression due to misalignment of the cervical spine.

The above conditions and symptoms can be alleviated by an appliance in accordance with the invention, as shown in FIG. 1. The appliance 1 has an anterior portion 2 and a posterior portion 6 connected by belts or straps 4. Each strap 4 proceeds through a retainer 5 attached at one side of the anterior portion 2. In the embodiment shown in FIG. 1, each strap 4 has a hook and loop surface, so that when the free end of each strap 4 is fed through the retainer 5, the strap 4 can be folded over on itself to set and fix a desired length of each strap 4 to accommodate a particular patient. The opposite end of each strap 4 is affixed in a suitable manner to the anterior portion 6.

As discussed in more detail below, the anterior portion 2 has a central opening 3 and is designed to be worn approximately over the abdomen, but without pressing directly on the central region of the abdomen.

As also shown in FIG. 1, the appliance 1 includes one or more pads 6A, which is/are temporarily attachable to the posterior portion 6 by a temporary attachment 7 such as hook-and-loop arrangement. The posterior portion 6 has padding or malting on the side thereof that is worn against the body. Although only one pad is shown in FIG. 1, multiple additional pads, corresponding to the pad 6A, can be used as needed to produce the necessary total thickness of the combination of the anterior portion 6 and pad or pads 6A, to produce the desired lordosis adjustment for alleviating symptoms or conditions of the type described above.

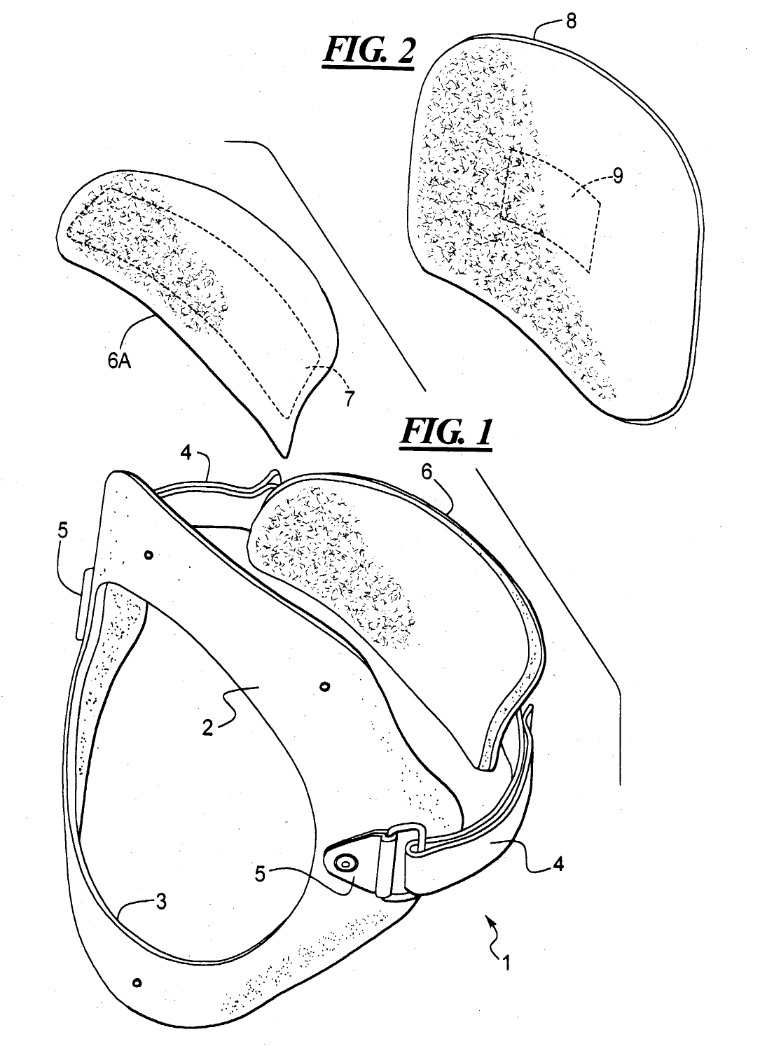

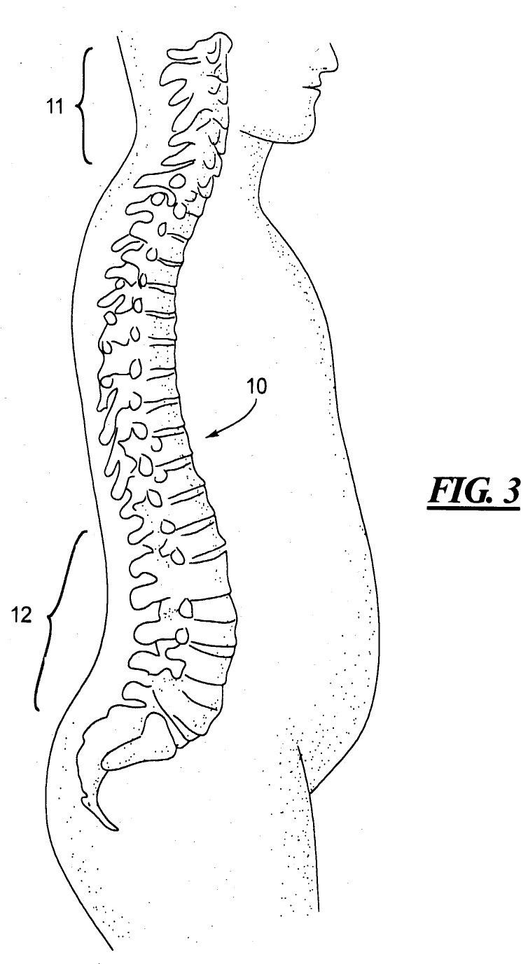

As also noted above, since it is not the purpose of the appliance 1 to serve as a brace or a support, the height of the posterior portion 6 is intentionally kept relatively short. For some conditions or symptoms, however, it may be desirable to slightly enlarge the height of the posterior portion 6, in which case a pad 8 such as shown in FIG. 2 can be used, which has a larger height than the pad 6A. Like pad 6A, the pad 8 can be attached to the posterior portion 6 with a temporary attachment 9, such as a hook-and-loop arrangement. Although not necessary, the posterior portion for use with the pad 8 can be enlarged to a size approximating the size of the pad 8. As noted above, however, it is not the overall size (area) of the pads which is effective for the intended purpose in the appliance 1, but rather their accumulated thickness to produce the desired lordosis adjustment.

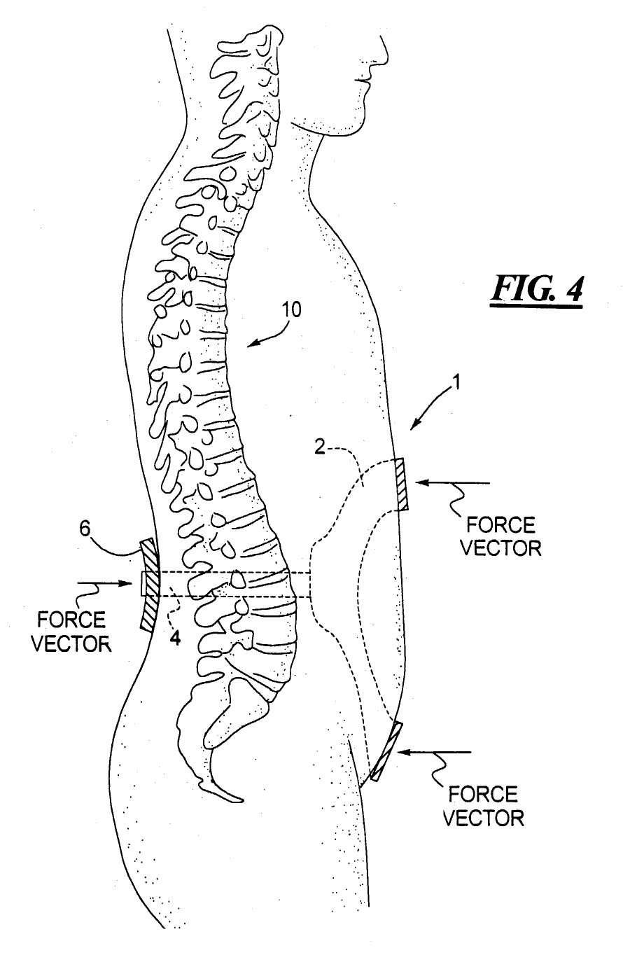

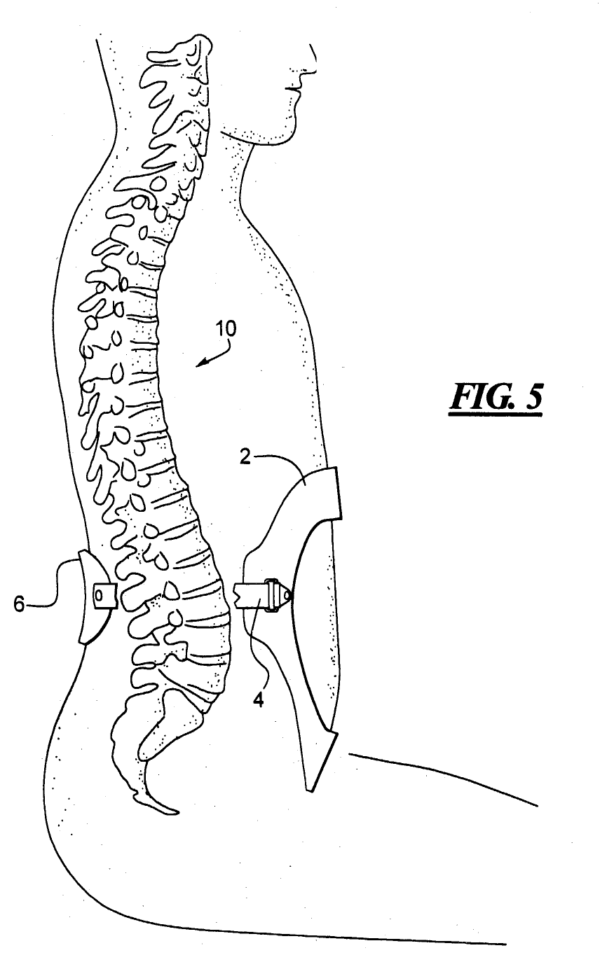

FIG. 3 is a schematic, sagittal view of a patient experiencing normal lordosis in the lumbar region 12 of the spinal column 10. Treatment of an absence of this lordosis in accordance with the inventive method, in the embodiment of wearing the appliance 1, is schematically indicated in FIG. 4, wherein the force vectors applied at upper and lower regions of the abdomen by the anterior portion 2, and at the lumbar region by the posterior portion 6, as illustrated. As can be seen in comparison to FIG. 3, healthy lordosis in the lumbar region 12 is restored by wearing the appliance 1, and this in turn slightly elevates the head on the neck, thereby restoring the cervical region to a neutral orientation on the upper body.

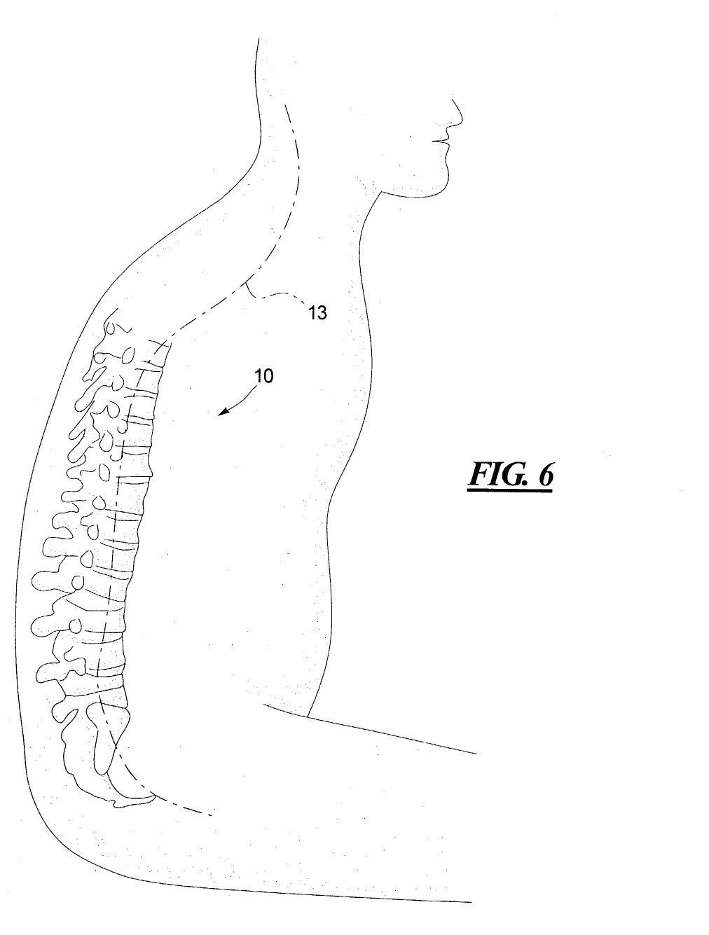

A similar situation is shown for a seated patient wearing the appliance 1 in FIG. 5. Without wearing such an appliance, the seated person would most likely exhibit the posture indicated by the dot and dash line 13 in FIG. 6. As can be seen in FIG. 5, proper posture, and neutral head and neck (cervical spine) orientation, are maintained and achieved by the appliance 1 worn by the seated person.

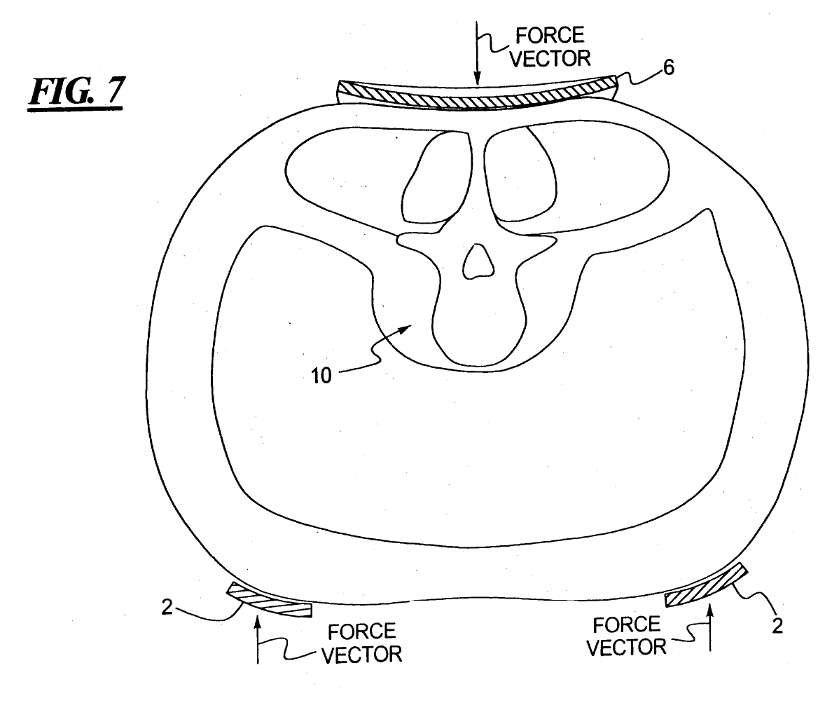

The application of the force vectors achieved by the appliance 1 is shown in a schematic axial view in FIG. 7. The force vectors indicated at the bottom of FIG. 7 occur approximately at the locations indicated by circles in the upper region of the anterior portion 2 in FIG. 1. The lower force vector (shown in FIG. 4) occurs approximately at the location indicated by the circle in the lower region of the anterior portion 2 in FIG. 1. As noted above, the force vectors applied by the anterior portion 2 in the upper region are at spaced-apart locations in the lower costal margin, and the lower force vector applied by the anterior portion 2 is approximately at the symphisis pubis. Such a three-point impact arrangement of the force vectors produced by the anterior portion 2 (in combination with the oppositely-directed force vector produced by the posterior portion 6) is specifically designed and positioned for achieving the desired lordosis adjustment for alleviating conditions and symptoms of the type described above.

The pad or pads 6A (and 8) are composed of a padding material which may be on a stiffer backing or carrier, such as a plastic carrier. The padding material should be sufficiently compressible so as to be comfortable for the patient to wear, however, it should not be so compressible that the desired force vectors cannot be produced when the appliance is worn with the straps 4 tightened to a comfortable length. Compressed fibers forming a mat of padding material are preferable, primarily because they are simplest to manufacture and maintain. Such a mat can simultaneously serve as part of the hook-and-loop temporary attachment. Other versions are possible, however, such as a fluid-filled cushion, or an inflatable cushion which can be inflated to the desired thickness as needed.

Although other modifications and changes may be suggested by those skilled in the art, it is the intention of the inventors to embody within the patent warranted hereon all changes and modifications as reasonably and properly come within the scope of their contribution to the art.

We claim as our invention:

1. A body-worn appliance for lordosis adjustment comprising:
an anterior portion having a substantially continuous, rigid frame with an open central region;
a rigid posterior portion;
two straps connecting said anterior portion and said posterior portion at opposite lateral sides thereof, said straps connecting said anterior portion to said posterior portion for positioning said posterior portion approximately at the L3 vertebra of a human patient and for positioning said anterior portion with an upper region of said frame approximately at a lower costal region of said patient and a lower portion of said frame approximately at the symphisis pubis of said patient;
said anterior portion and said posterior portion having substantially no therapeutic supporting and bracing capability; and
at least one pad that is removably attachable to said posterior portion to set a thickness of a combination of said pad and said posterior portion to adjust lordosis in said patient to produce a neutral cervical spine orientation of said patient.

2. An appliance as claimed in claim 1 wherein said posterior portion has a height substantially coextensive with one vertebra above and one vertebra below said L3 vertebra.

3. An appliance as claimed in claim 1 wherein said frame of said anterior portion has a shape to apply respective force vectors at two spaced-apart locations in said lower costal region and a location at said symphisis pubis.

4. An appliance as claimed in claim 1 wherein each of said straps has a first end attached to posterior portion and a free, second end opposite said first end, and a side surface having hook-and-loop fastening elements thereon, and wherein said anterior portion has two loops rigidly attached thereto at said lateral sides, the respective free ends of said straps being insertable through said loops so that each strap overlaps on itself for engaging said hook-and-loop fastening elements.

5. An appliance as claimed in claim 1 comprising a hook-and-loop fastening arrangement for temporarily attaching said at least one pad to said posterior portion.
